SEQUENCE LISTING (9 sequences)

SEQ ID NO: 1 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 2 - protein, 124 aa, synthetic construct
EVQLVESGGGVVQPGRSLRLSCAASGFTFSSYAMHWVRQAPGKGLEWVAAIWYDGSNQYYADSVKGRFTISRDDSTNTLYLQMNSLRAEDTALYYCAKSSYYDSSGYYFPYGYWGQGTLVTVSS

SEQ ID NO: 3 - protein, 8 aa, synthetic construct
GFTFSSYA

SEQ ID NO: 4 - protein, 8 aa, synthetic construct
IWYDGSNQ

SEQ ID NO: 5 - protein, 17 aa, synthetic construct
AKSSYYDSSGYYFPYGY

SEQ ID NO: 6 - protein, 119 aa, synthetic construct
EVQLVESGGGVVQPGRSLRLSCAASGFTFSSYAMHWVRQAPGKGLEWMGIIYPGDSDTRYSPSFQGHVTISRDDSTNTLYLQMNSLRAEDTATYYCARERGWQDAFDIWGQGTLVTVSS

SEQ ID NO: 7 - protein, 8 aa, synthetic construct
IYPGDSDT

SEQ ID NO: 8 - protein, 12 aa, synthetic construct
ARERGWQDAFDI

SEQ ID NO: 9 - protein, 8 aa, synthetic construct
DYKDDDDK

(19) United States
(12) Patent Application Publication
(10) Pub. No.: US 2025/0101134 A1
(43) Pub. Date: Mar. 27, 2025

(54) ANAPLASTIC LYMPHOMA KINASE ANTIBODIES AND METHODS OF USE THEREOF

(71) Applicants: The Children's Hospital of Philadelphia, Philadelphia, PA (US); UNIVERSITY OF PITTSBURGH - OF THE COMMONWEALTH SYSTEM OF HIGHER EDUCATION, Pittsburgh, PA (US)

(72) Inventors: Dimiter Dimitrov, Pittsburgh, PA (US); Chuan Chen, Pittsburgh, PA (US); Zehua Sun, Pittsburgh, PA (US); Yael Mosse, Penn Valley, PA (US)

(21) Appl. No.: 18/729,531

(22) PCT Filed: Jan. 24, 2023
(86) PCT No.: PCT/US23/61136

§ 371 Date: Jul. 17, 2024

Related U.S. Application Data
(60) Provisional application No. 63/302,639, filed on Jan. 25, 2022.

(51) Int. Cl. C07K 16/40; A61K 40/11; A61K 40/31; A61K 40/42; A61K 45/06; A61K 47/68; A61P 35/00

(52) U.S. Cl. CPC C07K 16/40; A61K 40/11; A61K 40/31; A61K 40/4251; A61K 45/06; A61K 47/68031; A61K 47/6871; A61P 35/00; A61K 2239/13; A61K 2239/47; C07K 2317/565

(57) ABSTRACT

Neuroblastoma (NB) remains a leading cause of childhood cancer morbidity and mortality. Heritable activating mutations are present in the anaplastic lymphoma kinase (ALK) oncogene and these same mutations are frequently somatically acquired during high-risk NB tumorigenesis. ALK has been established as a tractable molecular target in NB and provides the rationale for the clinical development of ALK inhibition therapy. Anti-ALK antibodies and antigen binding fragments thereof are provided along with methods of use thereof.

This application claims priority under 35 U.S.C. § 119(e) to U.S. Provisional Patent Application No. 63/302,639, filed Jan. 25, 2022. The foregoing application is incorporated by reference herein.

This invention was made with government support under grant number CA140198 awarded by the National Institutes of Health. The government has certain rights in the invention.

### FIELD OF THE INVENTION

The present invention relates to the fields of oncology. More specifically, the invention provides compositions and methods comprising anaplastic lymphoma kinase (ALK) antibodies for the treatment of cancer such as neuroblastoma.

### BACKGROUND OF THE INVENTION

Several publications and patent documents are cited throughout the specification in order to describe the state of the art to which this invention pertains. Each of these citations is incorporated herein by reference as though set forth in full.

Neuroblastoma (NB) remains a leading cause of childhood cancer morbidity and mortality. Heritable activating mutations are present in the anaplastic lymphoma kinase (ALK) oncogene and these same mutations are frequently somatically acquired during high-risk NB tumorigenesis. ALK has been established as a tractable molecular target in NB and provides the rationale for the clinical development of ALK inhibition therapy. Indeed, ALK is abundantly expressed on the cell surface of the vast majority of NBs and other pediatric malignancies while not being significantly expressed on normal tissues. Notably, the majority of activating ALK mutations are not sensitive to first generation drugs such as crizotinib. However, chemotherapy can sensitize ALK mutant NBs to crizotinib. Further, lorlatinib is the only ALK inhibitor that is effective against all activating mutations. Combinations of molecularly targeted agents (e.g., ceritinib and ribociclib for patients with ALK-driven NB) have shown synergistic activity. However, new therapeutics for the treatment of ALK expressing cancers are still needed.

### SUMMARY OF THE INVENTION

In accordance with the present invention, anti-anaplastic lymphoma kinase (ALK) antibodies and fragments thereof (e.g., antigen binding fragments) are provided. In certain embodiments, the anti-ALK antibody or fragment thereof binds amino acids 733-960 or amino acids 935-1038 of ALK. In certain embodiments, the antibody or fragment thereof comprises at least one complementarity determining region from VH20 or VH78. In certain embodiments, the antibody or fragment thereof comprises all three complementarity determining regions from VH20 or VH78. In certain embodiments, the antibody or fragment thereof comprises VH20 or VH78.

In accordance with another aspect of the present invention, immunoconjugates comprising an anti-ALK antibody or fragment thereof (e.g., antigen binding fragments) are provided. In certain embodiments, the immunoconjugate is an antibody-drug conjugate (ADC) comprising an anti-ALK antibody or fragment thereof. Bispecific T-cell engagers (BiTEs) comprising an anti-ALK antibody or fragment thereof (e.g., antigen binding fragments) are provided. Chimeric antigen receptors (CAR) and CAR-T cells comprising an anti-ALK antibody or fragment thereof (e.g., antigen binding fragments) are also provided.

In accordance with another aspect of the instant invention, methods for treating, inhibiting, and/or preventing (e.g., inhibiting the onset) of a cancer in a subject or patient are provided. In certain embodiments, the methods comprise administering an anti-ALK antibody or fragment thereof (e.g., antigen binding fragments) or a compound or cell comprising an anti-ALK antibody or fragment thereof (e.g., antigen binding fragments) to the subject or patient. In certain embodiments, the cancer expresses ALK, particularly on its surface. In certain embodiments, the cancer is neuroblastoma. In certain embodiments, the method further comprises administering a chemotherapeutic agent, radiotherapy, and/or an ALK inhibitor to the subject.

### BRIEF DESCRIPTION OF THE DRAWINGS

FIG. 1A is a schematic of the anaplastic lymphoma kinase (ALK) protein. FIG. 1B provides graphs of the binding of VH20 or VH9 to 293T cells or 293T cells expressing the G-rich domain (293T-ALK). FIG. 1C provides graphs of the binding of VH78 or VH5 to 293T cells or 293T cells expressing the G-T domain (293T-ALK).

FIGS. 2A-2D provide graphs showing aggregation of VH20 (FIG. 2A), VH9 (FIG. 2B), VH5 (FIG. 2C), and VH78 (FIG. 2D) in phosphate buffered saline (PBS) as determined by dynamic light scattering (DLS). FIG. 2A provides graphs of samples at day 0, day 1, day 4, day 7, and day 14 (top to bottom); FIG. 2B provides graphs of samples at day 0 and day 1 (top to bottom); FIG. 2C provides graphs of samples at day 0, day 1, day 4, and day 7 (top to bottom); and FIG. 2D provides graphs of samples at day 0, day 1, and day 4 (top to bottom).

FIGS. 3A-3D provide characterizations of the VHs. FIG. 3A provides graphs of ELISA assays with the indicated VH (⋅) and ALK-Fc, a fusion of the ALK extracellular domain fusion and human IgG1 Fc. Control (▴) without antibody also shown. FIG. 3B provides a graph of a size exclusion chromatography (SEC) assay for VH20. FIG. 3C provides a graph of the binding of ALK, VH20, and KTN0239-IgG. FIG. 3D provides graphs of the flow cytometry results of VH20 or control antibody binding to SK-N-AS, IMR32, or SY5Y-D3 cells.

FIG. 4 provides graphs of the cytotoxicity of CAR-T cells with VH9 (left), VH20 (left), VH5 (right), or VH78 (right) and 293T cells or 293T cells expressing ALK.

FIG. 5A provides a schematic of a VH20 bispecific T-cell engager (VH20-OKT3-Fc BiTE) and an image of an SDS-PAGE analysis of the VH20-OKT3-Fc BiTE in the presence or absence of beta-mercaptoethanol. FIG. 5B provides a graph of a size exclusion chromatography (SEC) assay for VH20-OKT3-Fc BiTE. FIG. 5C provides graphs of in vitro cytotoxicity assays of the VH20-OKT3-Fc BiTE or control (Iso) against the indicated cells.

FIG. 6A provides a graph of the internalization of labelled VH20 at various concentrations when cultured with 293T cells or 293T-ALK cells. FIG. 6B provides a graph of the cytotoxicity of monomethyl auristatin E (MMAE) or the antibody-drug conjugate VH20-Fc-MMAE cultured with 293T or 293T-ALK cells.

### DETAILED DESCRIPTION OF THE INVENTION

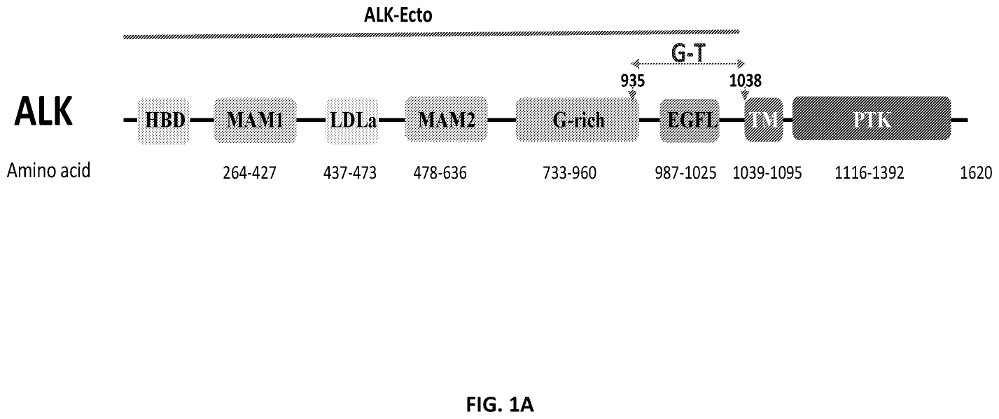

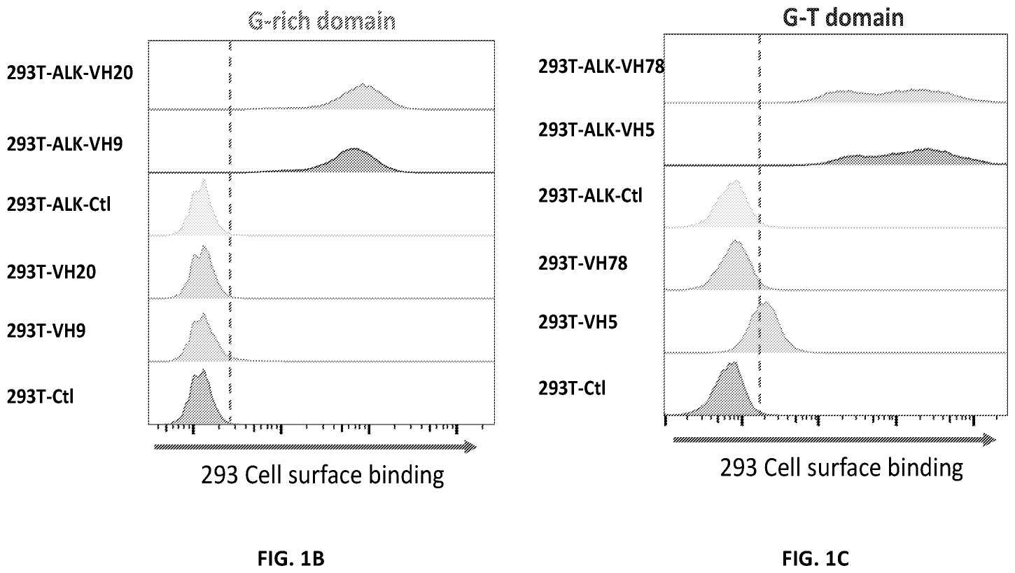

Human ALK protein is a 220 kDa cell surface receptor tyrosine kinase of the insulin receptor superfamily. FIG. 1A provides a schematic of the domains of ALK. ALK comprises two MAM domains (meprin/A5-protein/PTPmu) at amino acids 264-427 and 478-636, a low-density lipoprotein class A (LDLa) motif at amino acids 437-473, a glycine-rich region (G-rich) at amino acids 733-960, a transmembrane domain (TM) at amino acids 1039-1059, and a PTK (protein tyrosine kinase) domain at amino acids 1116-1392. ALK also comprises a heparin-binding domain (HBD) near the N-terminus (e.g., beginning at amino acid 19 after signal peptide) and an EGFL domain from amino acids 987-1025. ALK also comprises a TNFL module which may be combined with the G-rich region to form a globular TNFL-GR supradomain. The extracellular domain of ALK is the region of ALK N-terminal to the TM domain (e.g., amino acids 1-1038 or amino acids 19-1038). ALK amino acid and nucleotide sequences are provided, e.g., in GenBank Gene ID: 238; and GenBank Accession Nos. NM_004304.5 and NP_004295.2. In certain embodiments, the amino acid sequence of ALK comprises (SEQ ID NO: 1):

| | |
|---|---|
| MGAIGLLWLLPLLLSTAAVGSGMGTGQRAGSPAAGPPLQPRPELSYSRLQ | 50 |
| RKSLAVDFVVPSLFRVYARDLLLPPSSSELKAGRPEARGSLALDCAPLLR | 100 |
| LLGPAPGVSWTAGSPAPAEARTLSRVLKGGSVRKLRRAKQLVLELGEEAI | 150 |
| LEGCVGPPGEAAVGLLQFNLSELFSWWIRQGEGRLRIRLMPEKKASEVGR | 200 |
| EGRLSAAIRASQPRLLFQIFGTGHSSLESPTNMPSPSPDYFTWNLTWIMK | 250 |
| DSFPFLSHRSRYGLECSFDFPCELEYSPPLHDLRNQSWSWRRIPSEEASQ | 300 |
| MDLLDGPGAERSKEMPRGSFLLLNTSADSKHTILSPWMRSSSEHCTLAVS | 350 |
| VHRHLQPSGRYIAQLLPHNEAAREILLMPTPGKHGWTVLQGRIGRPDNPF | 400 |
| RVALEYISSGNRSLSAVDFFALKNCSEGTSPGSKMALQSSFTCWNGTVLQ | 450 |
| LGQACDFHQDCAQGEDESQMCRKLPVGFYCNFEDGFCGWTQGTLSPHTPQ | 500 |
| WQVRTLKDARFQDHQDHALLLSTTDVPASESATVTSATFPAPIKSSPCEL | 550 |
| RMSWLIRGVLRGNVSLVLVENKTGKEQGRMVWHVAAYEGLSLWQWMVLPL | 600 |
| LDVSDRFWLQMVAWWGQGSRAIVAFDNISISLDCYLTISGEDKILQNTAP | 650 |
| KSRNLFERNPNKELKPGENSPRQTPIFDPTVHWLFTTCGASGPHGPTQAQ | 700 |
| CNNAYQNSNLSVEVGSEGPLKGIQIWKVPATDTYSISGYGAAGGKGGKNT | 750 |
| MMRSHGVSVLGIFNLEKDDMLYILVGQQGEDACPSTNQLIQKVCIGENNV | 800 |
| IEEEIRVNRSVHEWAGGGGGGGGATYVFKMKDGVPVPLIIAAGGGGRAYG | 850 |
| AKTDTFHPERLENNSSVLGLNGNSGAAGGGGGWNDNTSLLWAGKSLQEGA | 900 |
| TGGHSCPQAMKKWGQETRGGFGGGGGGCSSGGGGGGYIGGNAASNNDPEM | 950 |
| DGEDGVSFISPLGILYTPALKVMEGHGEVNIKHYLNCSHCEVDECHMDPE | 1000 |
| SHKVICFCDHGTVLAEDGVSCIVSPTPEPHLPLSLILSVVTSALVAALVL | 1050 |
| AFSGIMIVYRRKHQELQAMQMELQSPEYKLSKLRTSTIMTDYNPNYCFAG | 1100 |
| KTSSISDLKEVPRKNITLIRGLGHGAFGEVYEGQVSGMPNDPSPLQVAVK | 1150 |
| TLPEVCSEQDELDFLMEALIISKFNHQNIVRCIGVSLQSLPRFILLELMA | 1200 |
| GGDLKSFLRETRPRPSQPSSLAMLDLLHVARDIACGCQYLEENHFIHRDI | 1250 |
| AARNCLLTCPGPGRVAKIGDFGMARDIYRASYYRKGGCAMLPVKWMPPEA | 1300 |
| FMEGIFTSKTDTWSFGVLLWEIFSLGYMPYPSKSNQEVLEFVTSGGRMDP | 1350 |
| PKNCPGPVYRIMTQCWQHQPEDRPNFAIILERIEYCTQDPDVINTALPIE | 1400 |
| YGPLVEEEEKVPVRPKDPEGVPPLLVSQQAKREEERSPAAPPPLPTTSSG | 1450 |
| KAAKKPTAAEISVRVPRGPAVEGGHVNMAFSQSNPPSELHKVHGSRNKPT | 1500 |
| SLWNPTYGSWFTEKPTKKNNPIAKKEPHDRGNLGLEGSCTVPPNVATGRL | 1550 |
| PGASLLLEPSSLTANMKEVPLFRLRHFPCGNVNYGYQQQGLPLEAATAPG | 1600 |
| AGHYEDTILKSKNSMNQPGP | 1620 |

In accordance with one aspect of the instant invention, anti-ALK antibodies and fragments thereof (e.g., antigen-binging fragments) are provided. The anti-ALK antibodies may be monoclonal or polyclonal. The anti-ALK antibodies may be bispecific. In certain embodiments, the antibody or fragment thereof is immunologically specific for human ALK. The anti-ALK antibodies or fragments thereof may recognize a linear epitope or a conformational epitope. In certain embodiments, the anti-ALK antibody or fragment thereof binds the G-rich domain of ALK (e.g., amino acids 733-960). In certain embodiments, the anti-ALK antibody or fragment thereof binds the G-T domain of ALK (e.g., amino acids 935-1038). In certain embodiments, the anti-ALK antibody or fragment thereof is immunologically specific for a polypeptide comprising amino acids 733-960 of ALK (e.g., SEQ ID NO: 1). In certain embodiments, the anti-ALK antibody or fragment thereof is immunologically specific for a polypeptide comprising amino acids 935-1038 of ALK (e.g., SEQ ID NO: 1). The above epitopes may be longer or shorter than the above identified sequences by 0, 1, 2, 3, 4, 5, 6, 7, 8, 9, 10, or more amino acids, particularly 1, 2, 3, 4, or 5 amino acids, at the N-terminus and/or C-terminus of the sequence. In certain embodiments, the above epitopes have at least 90%, 95%, 97%, 99%, or 100% homology or identity with SEQ ID NO: 1. Antibodies which bind the same epitope as an antibody provided herein are also encompassed by the instant invention.

In certain embodiments, the anti-ALK antibody or fragment thereof is immunologically specific for amino acids 733-960 of ALK. In certain embodiments, the anti-ALK antibody comprises VH20 or a fragment thereof. In certain embodiments, the anti-ALK antibody comprises a heavy chain (e.g., VH domain) comprising:

(SEQ ID NO: 2)
EVQLVESGGGVVQPGRSLRLSCAASGFTFSSYAMHWVRQAPGKGLEWVAA
IWYDGSNQYYADSVKGRFTISRDDSTNTLYLQMNSLRAEDTALYYCAKSS
YYDSSGYYFPYGYWGQGTLVTVSS,

wherein the underlined sequences are complementarity determining regions (CDRs). In certain embodiments, the anti-ALK antibody or fragment thereof comprises one, two, or all three CDRs of VH20 (e.g., as determined by IMGT, Chothia, Kabat, Martin (e.g., enhanced Chothia) or AHo numbering scheme). In certain embodiments, the anti-ALK antibody or fragment thereof comprises one, two, or all three CDRs of SEQ ID NO: 2. In certain embodiments, the anti-ALK antibody or fragment thereof comprises a heavy chain (e.g., VH domain) comprising one, two, or all three CDRs of SEQ ID NO: 2. In certain embodiments, the anti-ALK antibody or fragment thereof comprises one, two, or all three of: GFTFSSYA (SEQ ID NO: 3), IWYDGSNQ (SEQ ID NO: 4), and AKSSYYDSSGYYFPYGY (SEQ ID NO: 5). In another embodiment, the anti-ALK antibody or fragment thereof comprise an amino acid sequence having at least 90%, 95%, 97%, 99%, or 100% homology or identity with any of the sequences provided above (e.g., any of SEQ ID NOs: 2-5).

In certain embodiments, the anti-ALK antibody or fragment thereof is immunologically specific for amino acids 935-1038 of ALK. In certain embodiments, the anti-ALK antibody comprises VH78 or a fragment thereof. In certain embodiments, the anti-ALK antibody comprises a heavy chain (e.g., VH domain) comprising:

(SEQ ID NO: 6)
EVQLVESGGGVVQPGRSLRLSCAASGFTFSSYAMHWVRQAPGKGLEWMGI
IYPGDSDTRYSPSFQGHVTISRDDSTNTLYLQMNSLRAEDTATYYCARER
GWQDADFIWGQGTLVTVSS,

wherein the underlined sequences are complementarity determining regions (CDRs). In certain embodiments, the anti-ALK antibody or fragment thereof comprises one, two, or all three CDRs of VH78 (e.g., as determined by IMGT, Chothia, Kabat, Martin (e.g., enhanced Chothia) or AHo numbering scheme). In certain embodiments, the anti-ALK antibody or fragment thereof comprises one, two, or all three CDRs of SEQ ID NO: 6. In certain embodiments, the anti-ALK antibody or fragment thereof comprises a heavy chain (e.g., VH domain) comprising one, two, or all three CDRs of SEQ ID NO: 6. In certain embodiments, the anti-ALK antibody or fragment thereof comprises one, two, or all three of: GFTFSSYA (SEQ ID NO: 3), IYPGDSDT (SEQ ID NO: 7), and ARERGWQDAFDI (SEQ ID NO: 8). In another embodiment, the anti-ALK antibody or fragment thereof comprise an amino acid sequence having at least 90%, 95%, 97%, 99%, or 100% homology or identity with any of the sequences provided above (e.g., any of SEQ ID NOs: 3, 6-8).

Compositions comprising an anti-ALK antibody or fragment thereof of the instant invention and a carrier such as a pharmaceutically acceptable carrier are also encompassed herein. In certain embodiments, the composition comprises at least one anti-ALK antibody or antibody fragment and at least one carrier (e.g., a pharmaceutically acceptable carrier).

Nucleic acid molecules encoding an anti-ALK antibody or fragment thereof of the instant invention are also encompassed herein. In certain embodiments, the nucleic acid molecule encoding the anti-ALK antibody or fragment thereof comprises a nucleotide sequence encoding any of the amino sequences provided above. In certain embodiments, the nucleic acid molecules of the instant invention are contained within a vector, particularly an expression vector. The instant invention also encompasses cells comprising and, optionally, expressing a nucleic acid molecule of the instant invention (e.g., cells that secrete anti-ALK antibodies).

The antibody may be a synthetic or modified antibody (e.g., a recombinantly generated antibody; a chimeric antibody; a bispecific antibody; a humanized antibody; a camelid antibody; and the like). In certain embodiments of the instant invention, the antibody is a bispecific antibody.

The antibodies of the instant invention may be an antibody fragment. In a particular embodiment, the antibody fragment is an antigen binding fragment of the antibody. Antibody fragments include, without limitation, immunoglobulin fragments including, without limitation: single domain (Dab; e.g., single variable light or heavy chain domain), Fab, Fab′, F(ab′)₂, and F(v); and fusions (e.g., via a linker) of these immunoglobulin fragments including, without limitation: scFv, scFv₂, scFv-Fc, minibody, diabody, triabody, and tetrabody. The antibody may also be a protein (e.g., a fusion protein) comprising at least one antibody or antibody fragment. In certain embodiments, the antibody fragment is a single domain antibody (e.g., VH domain).

The antibodies of the instant invention may be further modified. For example, the antibodies may be humanized. In a particular embodiment, the antibodies (or a portion thereof) are inserted into the backbone of an antibody or antibody fragment construct (e.g., an antibody framework), particularly a human construct/framework. For example, the variable light domain and/or variable heavy domain of the antibodies of the instant invention or the CDRs contained therein may be inserted into another antibody construct or framework, particularly human. Methods for recombinantly producing antibodies are well-known in the art. Commercial vectors for antibody and antibody fragment constructs are available.

The antibodies of the instant invention may also be conjugated/linked to other components. Immunoconjugates comprising an anti-ALK antibody or fragment thereof of the instant invention are encompassed herein. For example, the antibodies or fragments thereof may be operably linked (e.g., covalently attached, optionally, through a linker) to at least one detectable agent (e.g., a radioactive atom (e.g., radioconjugate)), imaging agent, contrast agent, or therapeutic or drug (e.g., an antibody-drug conjugate). In certain embodiments, the anti-ALK antibody or fragment thereof are conjugated to a radionuclides (radioisotopes) such as, without limitation, positron-emitting isotopes and alpha-, beta-, gamma-, Auger- and low energy electron-emitters. The antibodies of the instant invention may also comprise at least one purification tag (e.g., a His-tag).

In accordance with the instant invention, antibody-drug conjugates (ADCs) are provided. ADCs are well known in the art (e.g., Polakis (2016) Pharmacol. Rev., 68:3-19). ADCs can be transported to target cells precisely due to the targeting capability of the antibodies, thereby effectively increasing local drug concentration at the target cells while greatly lowering drug concentration in other tissues or organs, thereby reducing reduced toxicity. Herein, the ADCs comprise an anti-ALK antibody or fragment thereof of the instant invention and a drug or therapeutic agent, particularly a cytotoxic agent. The drug and the antibody or fragment thereof may be directly conjugated or conjugated via a linker.

Generally, the drug or cytotoxic agent is a small molecule. As used herein, a “cytotoxic agent” is a substance that inhibits or prevents the function of cells and/or causes destruction and/or death of cells. Examples of cytotoxic agents include, without limitation: antimetabolites, alkylating agents, DNA minor groove binders, DNA intercalators, DNA crosslinkers, histone deacetylase inhibitors, nuclear export inhibitors, proteasome inhibitors, heat shock protein inhibitors, tyrosine kinase inhibitors, anti-mitotic agents, anthracyclines, cytoskeletal disruptors, taxanes, epothilones, toxins (e.g., diphtheria, exotoxin, ricin (e.g., ricin A), abrin, Pseudomonas exotoxin, saporin, etc.), topoisomerase I inhibitors, topoisomerase II inhibitors, nucleotide analogs, nucleotide precursor analogs, antibiotics (e.g., peptide antibiotics), platinum-based agents, maytansinoids (e.g., maitansine), auristatins (e.g., monomethyl auristatin E (MMAE)), doxorubicins, duocarmycins (e.g., thienoindoles), irinotecans, and pyrrolobenzodiazepines (PDB) or pyrrolobenzodiazepine dimers (see, e.g., Carlson (2012) Biotechnol. Healthc., 9(4): 28-31; WO 2005/040170).

In certain embodiments, the cytotoxic agent is a thienoindole. Thienoindoles (e.g., NMS-P528) are potent soluble duocarmycin analogs, which are DNA minor groove alkylating agents that are highly amenable to serve as antibody payloads (Valsasina, et al. (2014) Cancer Res., 74:822; Caruso, et al. (2018) Cancer Res. 78(13 Suppl): Abstract nr 734).

In certain embodiments, the cytotoxic agent is a pyrrolobenzodiazepine (PBD) dimer. In certain embodiments, the PBD dimers are conjugated to the antibody or fragment thereof through glycans on the CH2 domain. PBD dimers are potent cytotoxic DNA minor groove interstrand crosslinking agents that have demonstrated potency and efficacy in pediatric cancer models (Bosse, et al. (2012) Cancer Res., 72(8):2068-78; Wood, et al. (2013) Pediatr. Blood Cancer 60(11):1860-7).

The linkers of the ADCs may conjugate the antibody or fragment thereof at any chemically feasible location, preferably such that the activity of the antibody or fragment thereof (e.g., target binding) and the activity of the drug (e.g., killing of the cell) are not significantly adversely affected. For example, the drug may be conjugated to the antibody or fragment thereof via a lysine residue or a cysteine residue (e.g., in the antibody hinge or constant region or region other than the CDRs). Site-specific conjugation through glycans also does not affect binding and other properties of the antibody (Zhu, et al. (2014) MAbs 6(5):1190-200; Bosse, et al. (2017) Cancer Cell 32(3):295-309; Seaman, et al. (2017) Cancer Cell 31(4):501-15). C2-Azide-Galactose may be used as a substrate for the Fc-glycan modification. DBCO-PEG4-VA-drug (e.g., PDB) may be used as payload for the conjugation following a click chemistry-based approach (Baskin, et al. (2007) Proc. Natl. Acad. Sci., 104(43):16793-7).

The linker of the ADC may be a cleavable linker (e.g., so as to release the conjugated drug within the cell) (Kellogg, et al. (2011) Bioconjug. Chem., 22(4):717-27; Polson, et al. (2011) Expert Opin. Investig. Drugs 20(1):75-85). For example, an acid-labile linker (e.g., hydrazone linker), lysosomal or peptidase-sensitive linker (e.g., short peptidyl linkers typically comprising a dipeptide such as Val-Cit, Val-Lys, Val-Ala, Lys-Lys, or Ala-Val), or disulfide-containing linker may be used. In certain embodiments, the antibody is conjugated to the drug with a peptidic cleavable drug linker and a self-immolative spacer, allowing for a protective moiety that requires cleavage of two chemical bonds prior to activation, through partial reduction of cysteine residues, yielding a desirable drug-antibody ratio (DAR) (e.g., of ˜2.7). One advantage of the peptide linker is the requirement for intracellular enzymatic cleavage by lysosomal proteases for the release of the cytotoxic payload.

Bifunctional coupling agents or crosslinkers may be used to conjugate the drug to the antibody or fragment thereof. Crosslinkers may comprise, without limitation, one or more carboxyl-to-amine reactive groups (e.g., carbodiimide), amine-reactive groups (e.g., NHS ester, imidoester), sulfhydryl-reactive groups (e.g., maleimide, haloacetyl, pyridyldisulfide), aldehyde-reactive groups (e.g., hydrazide, alkoxyamine), and hydroxyl-reactive groups (e.g., isocyanate).

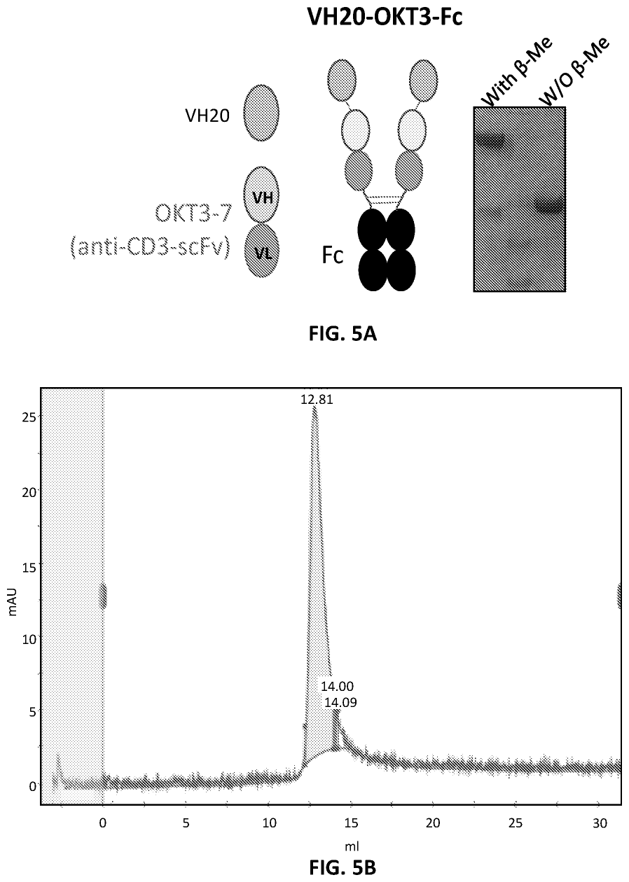

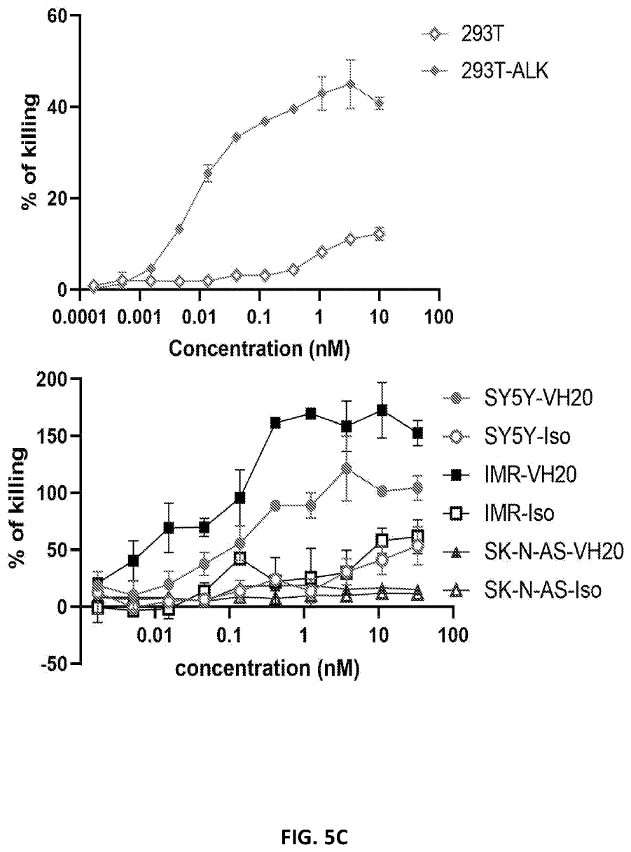

In accordance with the instant invention, bispecific T-cell engangers (BiTEs) are provided. FIG. 5A provides a schematic of an example of a BiTE. Generally, BiTEs have two antigen-binding domains, one of which binds to a T-cell antigen and the second of which binds to an antigen present on the surface of a target (see, e.g., WO 05/061547; Baeuerle et al. (2008) Drugs of the Future 33:137-147; Bargou et al. (2008) Science 321:974-977). BiTEs of the instant invention may comprise two binding sites, the first binding site comprises an anti-ALK antibody or fragment thereof as described herein and the second binding site comprising an antibody or fragment thereof that specifically binds to a T cell (e.g., a cell surface target such as CD3). In certain embodiments, the BiTE is a single polypeptide chain molecule. In certain embodiments, the BiTE comprises an Fc region. In certain embodiments, the antibody or fragment thereof that specifically binds to a T cell is an anti-CD3 antibody or fragment thereof. In certain embodiments, the anti-CD3 antibody or fragment thereof is an scFv. In certain embodiments, the anti-CD3 antibody or fragment thereof is OKT3.

The present invention also encompasses the use of sequences (e.g., the CDR or VH sequences) of an anti-ALK antibody or fragment thereof described herein in the preparation of a chimeric antigen receptor, which may be for use in CAR-T technology. As used herein, “chimeric antigen receptor” or “CAR” refers to a hybrid polypeptide comprising an antigen-binding domain (e.g., an antigen-binding portion of an antibody) linked to a cell signaling and/or cell activation domain. In certain embodiments, the chimeric antigen receptor of the instant invention comprises an ectodomain (extracellular domain), a transmembrane domain, and an endodomain (cytoplasmic or intracellular domain). The ectodomain of the chimeric antigen receptor of the instant invention comprises an anti-ALK antibody or fragment thereof. In certain embodiments, the antibody or fragment thereof comprises a Fab or a scFv. The antibody or an antigen-binding fragment of the ectodomain may be linked to the transmembrane domain via an amino acid linker/spacer (e.g., about 1 to about 100 amino acids). The ectodomain may also comprise a signal peptide (e.g., an endoplasmic reticulum signal peptide).

The transmembrane domain of the chimeric antigen receptor of the instant invention may be any transmembrane domain. In a particular embodiment, the transmembrane domain is a hydrophobic alpha helix that spans the cell membrane. Typically, the transmembrane domain is from the same protein as the endodomain. Examples of transmembrane domains include, without limitation, transmembrane domains from T-cell receptor (TCR), CD28, CD3, CD45, CD4, CD5, CD8, CD9, CD16, CD22, CD33, CD37, CD64, CD80, CD86, CD134, CD137, or CD154. In a particular embodiment, the transmembrane domain is from CD3-ζ or CD28.

The endodomain of the chimeric antigen receptor of the instant invention comprises at least one signaling or activation domain (e.g., a signaling domain comprising one or more immunoreceptor tyrosine-based activation motifs (ITAMs)). The signaling or activation domain is activated by antigen binding to the ectodomain and leads to the activation of the T cells. Signaling or activation domains include, without limitation, the signaling or activation domain (e.g., endodomain/cytoplasmic domain or fragment thereof) from CD3, LIGHT, lymphocyte function-associated antigen 1 (LFA-1), CD2, CD28, ICOS, CD30, CD7, NKG2C, CD40, PD-1, OX40, CD18, CD27, B7-H3, 4-1BB (CD137), OX40, CD40, and NKG2C. In certain embodiments, the endodomain comprises the cytoplasmic domain or fragment thereof of CD3-ε, CD3-γ, or CD3-ζ chain. In certain embodiments, the endodomain comprises the signaling domains of CD3-ζ, CD28, 4-1BB, and/or OX40. In certain embodiments, the endodomain comprises the signaling domains of CD3-ζ, CD28, and OX40. In certain embodiments, the endodomain comprises the signaling domains of CD3-ζ, CD28, and 4-1BB. In certain embodiments, the endodomain comprises the 4-1BB signaling domain.

Nucleic acid molecules encoding the CAR (e.g., vectors) of the instant invention may be transferred into the desired target cell (e.g., T cell) by any physical, chemical, or biological means.

The methods of the instant invention encompass administering a nucleic acid (DNA or RNA) encoding a CAR to the subject. In a particular embodiment, the method comprises administering T cells (e.g., T cell, cytotoxic T cell, and/or natural killer) comprising a nucleic acid encoding CAR to the subject. The T cells may be autologous. For example, the methods may comprise transducing T cells ex vivo with a nucleic acid encoding a chimeric antigen receptor of the instant invention (e.g., an integrating or non-integrating vector for the expression of the chimeric antigen receptor). The methods of the instant invention may further comprise obtaining the T cells from the subject.

The antibody molecules of the invention may be prepared using a variety of methods known in the art. Polyclonal and monoclonal antibodies may be prepared as described in Current Protocols in Molecular Biology, Ausubel et al. eds. Antibodies may be prepared by chemical cross-linking, hybrid hybridoma techniques and by expression of recombinant antibody fragments expressed in host cells, such as bacteria or yeast cells. In one embodiment of the invention, the antibody molecules are produced by expression of recombinant antibody or antibody fragments in host cells. The nucleic acid molecules encoding the antibody may be inserted into expression vectors and introduced into host cells. The resulting antibody molecules are then isolated and purified from the expression system. The antibodies optionally comprise a purification tag by which the antibody can be purified.

The purity of the antibody molecules of the invention may be assessed using standard methods known to those of skill in the art, including, but not limited to, ELISA, immunohistochemistry, ion-exchange chromatography, affinity chromatography, immobilized metal affinity chromatography (IMAC), size exclusion chromatography, polyacrylamide gel electrophoresis (PAGE), western blotting, surface plasmon resonance and mass spectroscopy.

In accordance with another aspect of the instant invention, methods for the inhibition, treatment, and/or prevention of cancer are provided. In certain embodiments, the cancer is an ALK expressing cancer. In certain embodiments, the cancer is a rhabdmyosarcoma, medulloblastoma, glioma, Ewing sarcoma, or neuroblastoma. In certain embodiments, the cancer is neuroblastoma. The methods comprise administering an anti-ALK antibody or fragment thereof of the instant invention to a subject in need thereof. The anti-ALK antibodies may be administered in a composition further comprising a pharmaceutically acceptable carrier. The anti-ALK antibody or fragment thereof may be administered as an immunoconjugate. The anti-ALK antibody or fragment thereof may be administered as an ADC. The anti-ALK antibody or fragment thereof may be administered as a BiTE. The anti-ALK antibody or fragment thereof may be administered as a CAR. The anti-ALK antibody or fragment thereof may be administered as a CAR-T cell.

In certain embodiments, the cancer is characterized by at least one mutation in ALK, particularly one which leads to increased activity of ALK (e.g., increased kinase activity) and/or an increased ALK copy number compared to normal human cells. In certain embodiments, the ALK comprises at least one mutation at position P36, P157, V198, G640, L684, G718, D993, L1204, I1170, A1200, L1204, F1245, G1128, R1192, R1275, D1091, M1166, I1171, F1174, F1245, or I1250. In certain embodiments, the ALK comprises at least one mutation at position G1128, R1192, R1275, D1091, M1166, I1171, F1174, F1245, or I1250. In certain embodiments, the ALK comprises at least one mutation at position G1128, R1192, and R1275. In certain embodiments, the ALK comprises at least one mutation selected from the group consisting of P36S, P157S, V198M, G640R, L684M, G718F, G718S, D993G, L1204F, I1170S, A1200V, L1204F, F1245I, G1128A, R1192P, R1275Q, D1091N, M1166R, I1171N, F1174I, F1174L, F1245C, F1245V, I1250T, T1151M, I1170S, F1174C, L1196M, F1245I, R259H, M770I, E1407K, E1433del, R1464G, G1494R, and A1553P. In certain embodiments, the ALK comprises at least one mutation selected from the group consisting of G1128A, R1192P, R1275Q, D1091N, M1166R, I1171N, F1174I, F1174L, F1245C, F1245V, and I1250T. In certain embodiments, the ALK comprises at least one mutation to amino acid R1275 and/or F1174, particularly at least one of R1275Q, F1174I, and F1174L. In certain embodiments, the cancer may be resistant to an ALK inhibitor (e.g., crizotinib).

The composition may further comprise at least one other therapeutic agent against the cancer. In certain embodiments, the other therapeutic agent is a chemotherapeutic agent and/or an ALK inhibitor (e.g., ALK siRNA and/or antisense molecule, crizotinib, TAE684 (Novartis), CEP-14083 (Cephalon), lorlatinib, ceritinib, or ribociclib). Alternatively, the other therapeutic agent may be contained within a separate composition(s) with at least one pharmaceutically acceptable carrier. The separate composition can be administered at the same time (e.g., simultaneously) and/or at different times (e.g., sequentially) as the composition comprising the anti-ALK antibody or fragment thereof. For example, the other therapeutic agent(s) may be administered separately (before, after, or at the same time as the anti-ALK antibody or fragment thereof) or in the same composition. In certain embodiments, the anti-ALK antibody or fragment thereof is administered with radiotherapy at the same time (e.g., simultaneously) and/or at different times (e.g., sequentially). The composition(s) comprising at least one anti-ALK antibody and/or the composition(s) comprising at least one other therapeutic agent may be contained within a kit.

The compositions of the present invention can be administered by any suitable route, for example, by injection (e.g., for local or systemic administration), parenterally, subcutaneously, orally (e.g., liquid or pill/capsule/tablet form), topically, pulmonarily, intravenously, intraperitoneally, intrathecally, epidurally, intramuscularly, intradermally, nasally, or other modes of administration. In a particular embodiment, the compositions are administered by injection (e.g., parenterally, subcutaneous, or into the bloodstream (e.g., intravenously)). The compositions may be administered directly to the site of the cancer. The compositions may be administered intravenously or orally. The compositions comprising the antibodies of the invention may be conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. Selection of a suitable pharmaceutical preparation depends upon the method of administration chosen. The concentration of the antibodies in the chosen medium may be varied and the medium may be chosen based on the desired route of administration of the pharmaceutical preparation. Except insofar as any conventional media or agent is incompatible with the agents to be administered, its use in the pharmaceutical preparation is contemplated. The pharmaceutical composition of the present invention can be prepared, for example, in liquid form, or can be in dried powder form (e.g., lyophilized).

Pharmaceutical compositions containing agents of the present invention as the active ingredient in intimate admixture with a pharmaceutical carrier can be prepared according to conventional pharmaceutical compounding techniques. The carrier may take a wide variety of forms depending on the form of preparation desired for administration. In preparing the antibody in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets).

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

The dose and dosage regimen of the antibodies according to the invention that is suitable for administration to a particular patient may be determined by a physician considering the patient's age, sex, weight, general medical condition, and the specific condition and severity thereof for which the agent is being administered. The physician may also consider the route of administration of the antibodies, the pharmaceutical carrier with which the antibodies may be combined, and the antibodies' biological activity. The appropriate dosage unit for the administration of the agents of the invention may be determined by evaluating the toxicity of the agents in animal models. Appropriate dosage unit may also be determined by assessing the efficacy of the agents in combination with other standard drugs.

The compositions comprising the agents of the instant invention may be administered at appropriate intervals, for example, at least once a day or more until the pathological symptoms are reduced or alleviated, after which the dosage may be reduced to a maintenance level. The appropriate interval in a particular case would normally depend on the condition of the patient.

### Definitions

The following definitions are provided to facilitate an understanding of the present invention:

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, excipient, auxilliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers. Suitable pharmaceutical carriers are described, for example, in “Remington's Pharmaceutical Sciences” by E. W. Martin.

An “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule (e.g., antigen-binding fragment), and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

As used herein, the term “subject” refers to an animal, particularly a mammal, particularly a human.

A “therapeutically effective amount” of a compound or a pharmaceutical composition refers to an amount effective to prevent, inhibit, treat, or lessen the symptoms of a particular disorder or disease. The treatment of a disease or disorder herein may refer to curing, relieving, and/or preventing the disease or disorder, the symptom(s) of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

The term “isolated” refers to the separation of a compound from other components present during its production or from its natural environment. “Isolated” is not meant to exclude artificial or synthetic mixtures with other compounds or materials, or the presence of impurities that do not substantially interfere with the fundamental activity, and that may be present, for example, due to incomplete purification, or the addition of stabilizers.

As used herein, the term “small molecule” refers to a substance or compound that has a relatively low molecular weight (e.g., less than 2,000). Typically, small molecules are organic, but are not proteins, polypeptides, or nucleic acids.

Chemotherapeutic agents are compounds that exhibit anticancer activity and/or are detrimental to a cell (e.g., a toxin). Suitable chemotherapeutic agents include, but are not limited to: toxins (e.g., saporin, ricin, abrin, ethidium bromide, diptheria toxin, and Pseudomonas exotoxin); taxanes; alkylating agents (e.g., temozolomide, nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes (e.g., cisplatin, carboplatin, tetraplatin, ormaplatin, thioplatin, satraplatin, nedaplatin, oxaliplatin, heptaplatin, iproplatin, transplatin, and lobaplatin); bioreductive alkylators such as mitomycin, procarbazine, dacarbazine and altretamine); DNA strand-breakage agents (e.g., bleomycin); topoisomerase II inhibitors (e.g., amsacrine, menogaril, amonafide, dactinomycin, daunorubicin, N,N-dibenzyl daunomycin, ellipticine, daunomycin, pyrazoloacridine, idarubicin, mitoxantrone, m-AMSA, bisantrene, doxorubicin (adriamycin), deoxydoxorubicin, etoposide (VP-16), etoposide phosphate, oxanthrazole, rubidazone, epirubicin, bleomycin, and teniposide); DNA minor groove binding agents (e.g., plicamydin); antimetabolites (e.g., folate antagonists such as methotrexate and trimetrexate); pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); anthracyclines; and tubulin interactive agents (e.g., vincristine, vinblastine, and paclitaxel (Taxol®))

The following example is provided to illustrate certain embodiments of the invention. It is not intended to limit the invention in any way.

### EXAMPLE

Recent comprehensive genomic analyses have yielded the sobering conclusion that actionable recurrent somatic mutations are rare in pediatric cancers, raising the prospect that it is necessary to move beyond small molecules targeting mutant kinases in order to substantially improve outcomes using precisely developed therapies. There is an urgent need for ALK-targeted antibodies as a therapeutic approach in neuroblastoma and other ALK-expressing childhood tumors. Significantly, ALK is expressed on the surface of neuroblastoma cells, but not in normal tissue (Carpenter, et al. (2012) Oncogene 31(46):4859-67). Notably, ALK is also differentially expressed at high levels in subsets of rhabdmyosarcomas, medulloblastomas, gliomas, and Ewing sarcomas (Mosse, Y. P. (2016) Clin. Cancer Res., 22(3):546-52; Corao (2009) Pediatric Dev. Pathol., 12(4):275-83; Pillay, et al. (2002) Histopathology 41(5):461-7; Yoshida et al. (2013) Mod. Pathol., 26(6):772-81). This indicates that ALK is also a target for immunotherapy. For example, antibody-drug conjugates (ADC) against the ALK extracellular region can be used for immunotherapeutic targeting of ALK. ADCs are a rapidly growing class of anti-cancer drugs that combine the targeting properties of antibodies (e.g., monoclonal antibodies) that are specific to tumor cell-surface proteins (Sliwkowski, et al. (2013) Science 341(6151):1192-8) with the anti-tumor effects of potent cytotoxic drugs (Teicher, et al. (2011) Clin. Cancer Res., 17(20):6389-97).

FIG. 1A provides a schematic of ALK. MAM1, LDLa, MAM2, G-rich, G-T domains and full-length ALK extracellular domain (ALK-Ecto) were expressed separately for phage panning. Briefly, 293T and 293T-ALK cells were stained with different VH antibodies at a concentration of 1 μM in 200 mL PBS for 30 minutes on ice. The cells were washed with 1 mL PBS three times and incubated with anti-FLAG-PE antibody (1:200, BioLegend) for 30 minutes on ice. The FLAG tag comprises the sequence: DYKDDDDK (SEQ ID NO: 9). The stained cells were then washed with 1 mL PBS three times. Antibody binding was detected on a BD™ LSR II (BD Biosciences, San Jose, CA). Fluorescence activated cell sorting (FACS) data analysis was performed using FlowJo_V10_CL.

FIGS. 1B and 1C show the flow cytometry results with various VHs binding to ALK extracellular domain expressed on the cell surface of 293T cells (293T-ALK). VH9 and VH20 were identified with the G-rich domain antigen while VH5 and VH78 were identified with the G-T domain antigen. All four VHs showed good binding with 293T-ALK cells while showing no significant non-specific binding with 293T cells.

The 4 VHs were studied by dynamic light scattering (DLS). Briefly, VH antibodies were buffer changed with PBS and filtered with a 0.22 μM filter.

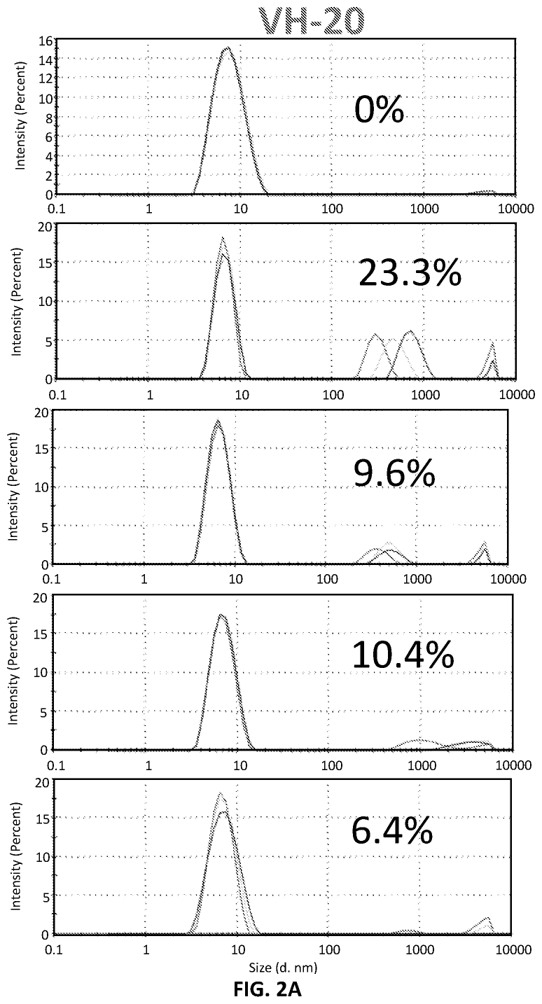

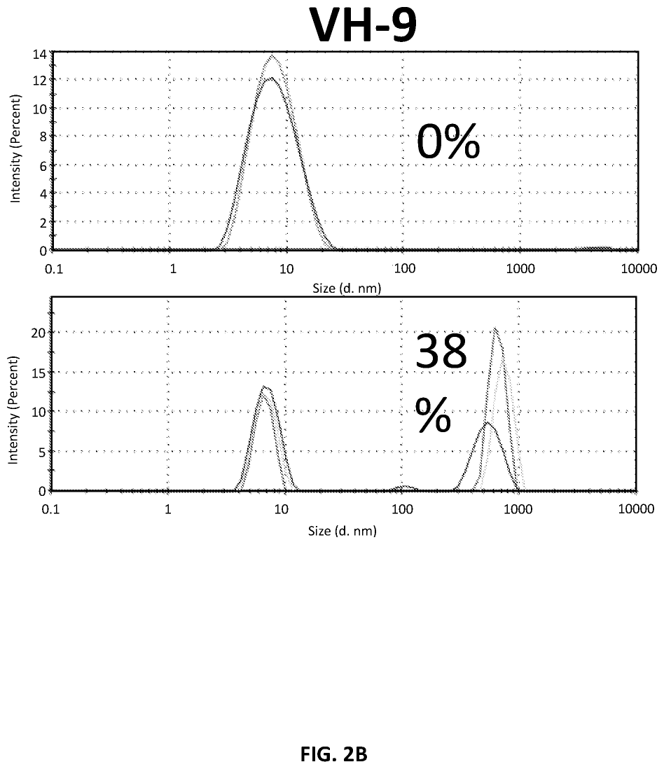

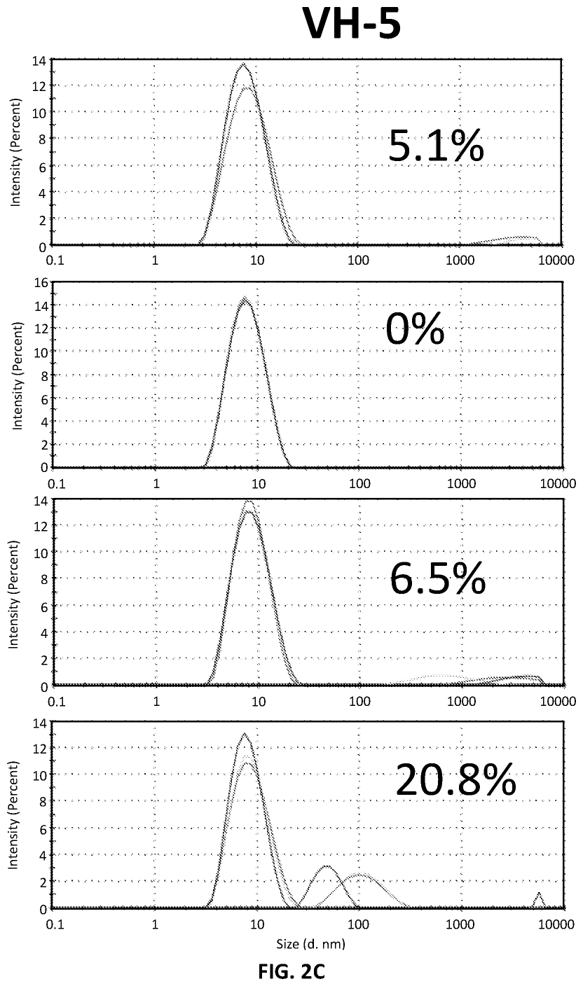

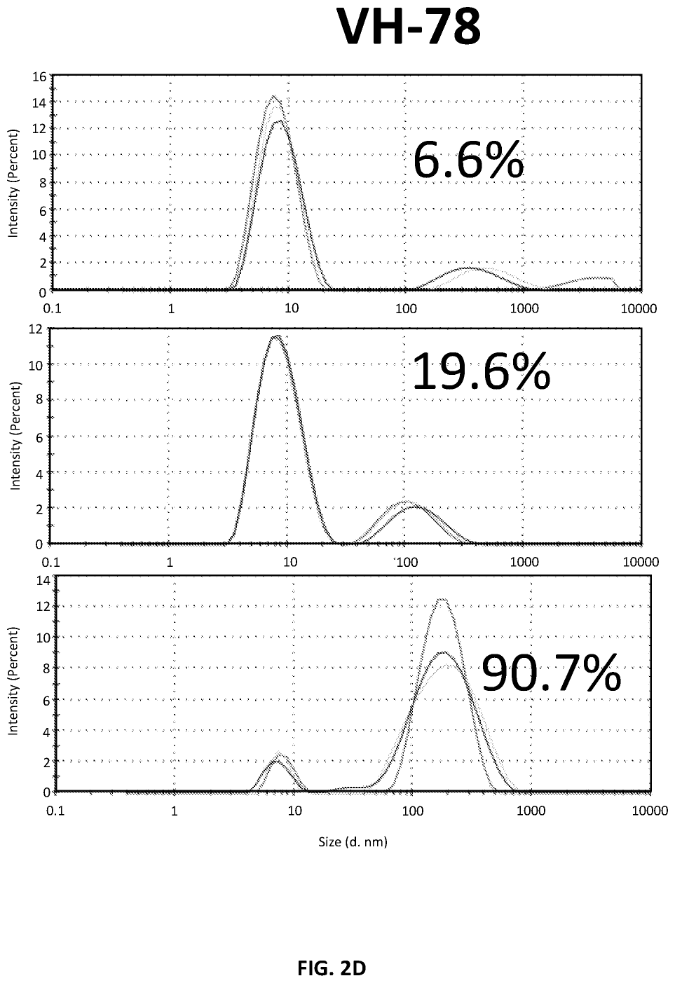

Antibody concentration was adjusted to 5 mg/mL. 500 μL samples were incubated at 37° C. for DLS. Samples were measured at day 0, day 1, day 4, day 7, and day 14 on Zetasizer Nano-ZS ZEN 3600 (Malvern Instruments Limited; Malvern, UK) to determine the size distributions of protein particles. As seen in FIGS. 2A-2D, VH20 and VH5 showed lower aggregation then VH9 and VH78.

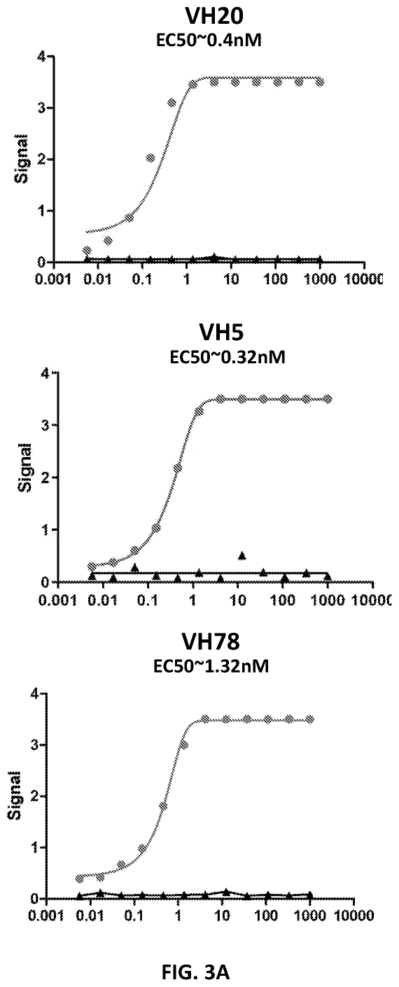

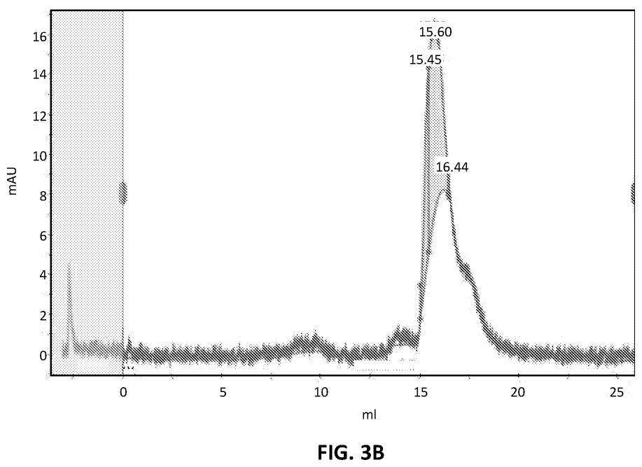

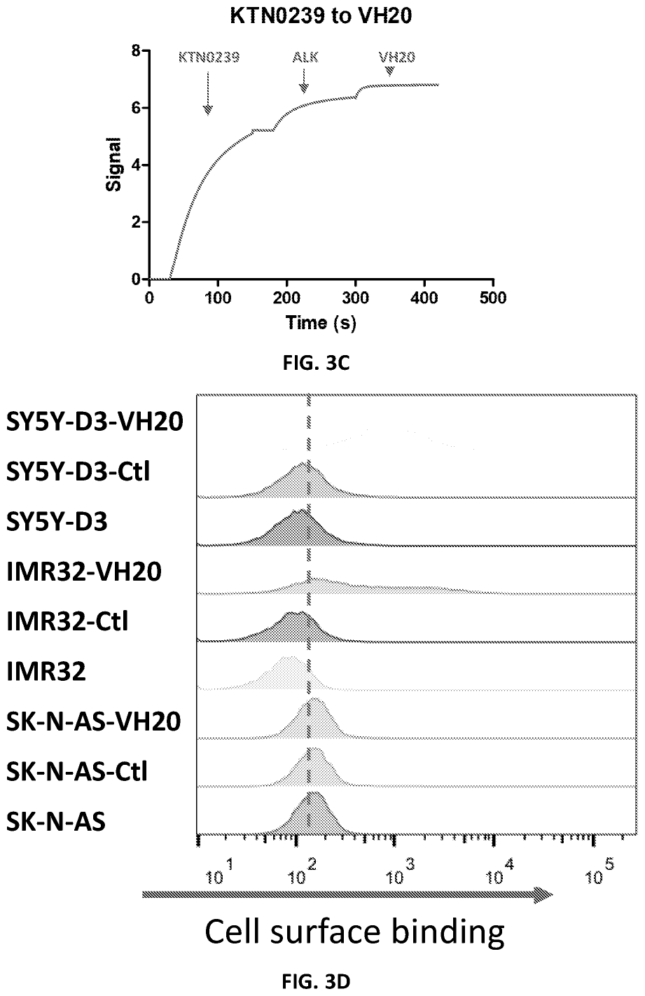

The VHs were further characterized. First, the binding affinities of the VHs were studied in an ELISA assay using a fusion protein comprising the ALK extracellular domain fused to the human IgG1 Fc (ALK-Fc). ELISA plates (Corning 3690; Tewksbury, MA) were coated with 50 μl antigen (5 μg/ml diluted with 1×PBS) at 4° C. overnight. The next day, blocking was performed with 150 μL 5% milk-PBS (Bio-RAD; Hercules, CA) at room temperature for two hours. Then, the plates were washed with 0.05% PBST three times. 3-fold serial diluted VH antibodies (in 5% PBS-Milk) was added into each well and incubated at room temperature for 1 hour and then washed with PBST 4 times. Next, 50 μL anti-Flag-HRP (1:1000 dilution in 5% PBS-Milk, Thermo; Waltham, MA) was added into each well and incubated at room temperature for 1 hour and then washed 5 times with PBST. 50 μL 3,3′,5,5′-tetramethylbenzidine (TMB) substrate (Sigma; St. Louis, MO) was added into each well and color was allowed to develop for 1-2 minutes before stopping with with 50 μL H₂SO₄ (1M, Sigma). Plates were read at 450 nm absorbance. The ELISA results were analyzed using GraphPad Prism 9.0.2. As seen in FIG. 3A, VH20 showed an EC₅₀ for ALK of about 0.4 nM, VH5 showed an EC₅₀ for ALK of about 0.32 nM, and VH78 showed an EC₅₀ for ALK of about 1.32 nM.

VH20 was also studied by size exclusion chromatography (SEC). 200 μL (1 μg/mL) filtered samples were used for analysis. A Superdex® 200 Increase 10/300 GL column (GE Healthcare, Cat. No. 28990944) was used for the SEC. The column was calibrated with protein molecular mass standards of ferritin (440 kDa), aldolase (158 kDa), conalbumin (75 kDa), ovalbumin (44 kDa), carbonic anhydrase (29 kDa), and ribonuclease A (13.7 kDa). Protein was eluted by Dulbecco's phosphate-buffered saline (DPBS) buffer at a flow rate of 0.5 mL/minute. As seen in FIG. 3B, VH20 was determined to be a dimer by SEC.

The ability of VH20 to compete with KTN0239 was also studied. KTN0239 is a humanized variant of KTN0125 (Sano et al. (2016) Cancer Res., 76(14 Suppl):Abstract2690) and can bind the G rich domain of ALK. A Protein A biosensor (18-5010, BLItz® system, ForteBio; Freemont, CA) was used to immobilization KTN0239-IgG. DPBS (pH=7.4) was used for the baseline and determine dissociation. The detection conditions used were (I) baseline 30 seconds; (II) loading KTN0239-IgG for 120 seconds; (III) baseline 30 seconds; (IV) association for 120 seconds with ALK; and (V) association for 120 seconds with VH20. As seen in FIG. 3C, VH20 is not in competition with KTN0239-IgG.

The ability of VH20 bind ALK expressing cells was also studied by FACS. SK-N-AS (negative cell line), IMR32 (positive cell line), SY5Y-D3 (positive cell line) cells were stained with VH20 at a concentration of 1 μM in 200 μL PBS for 30 minutes on ice. The cells were washed with 1 mL PBS three times and incubated with anti-Flag-PE antibody (1:200, BioLegend) for 30 minutes on ice. The cells were again washed with 1 mL PBS three times. Antibody binding was detected on a BD™ LSR II (San Jose, CA). FACS data analysis was performed using FlowJo_V10_CL. As seen in FIG. 3D, VH20 was capable of binding cell surface ALK on IMR32 and SY5Y-D3 cells, but not the negative control of SK-N-AS cells.

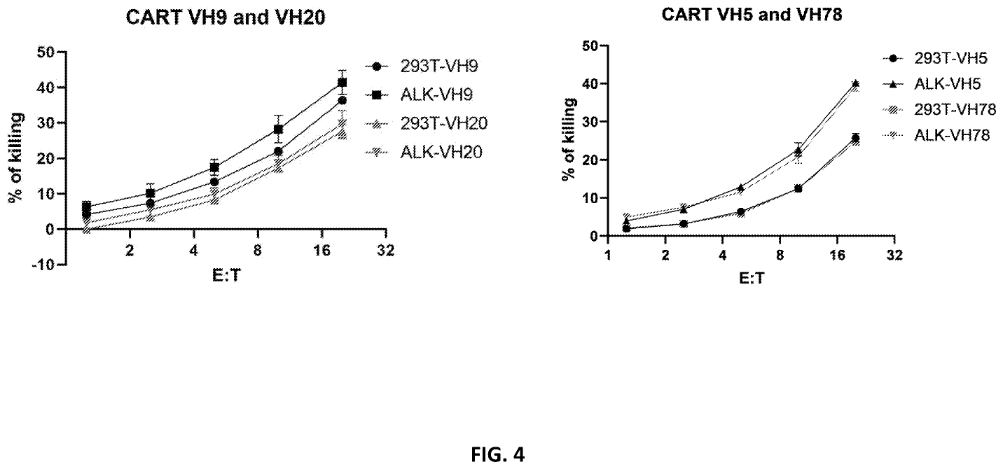

The cytotoxicity of anti-ALK CAR-T cells against 293T and 293T-ALK cells was also studied. Anti-ALK CAR-T cells (effector cells) were incubated with 293T and 293T-ALK cells at the effector: target ratios of 1.25:1, 2.5:1, 5:1, 10:1 and 20:1 for 48 hours in a 96-well cell culture plate (Corning). The co-cultures of CAR-T cells with 293T cells were used as negative controls. Cytotoxicity was determined by detecting specific lactate dehydrogenase (LDH) released into the medium from the target cells with cytotoxicity LDH detection kit (Promega) according to the manufacturer's instructions. Percent cytotoxicity was calculated with the following formula: Cytotoxicity (%)=(Experimental lysis−Effector spontaneous lysis−Target spontaneous lysis)/(Target maximum lysis−Target spontaneous lysis)×100%. Anti-ALK CAR-T cells were co-cultured with 293T cells as negative controls. As seen in FIG. 4, greater cytotoxicity was detected with VH5 and VH78.

A VH20 bispecific T-cell engager (VH20-OKT3-Fc BiTE) was also synthesized and characterized. FIG. 5A provides a schematic of human VH20-OKT3-Fc BiTE. VH20, OKT3-7 scFV and IgG1 Fc were fused to generate the BiTE. FIG. 5A also provides the size of the BiTE on SDS-PAGE with (reducing) or without beta-mercaptoethanol. Proteins (comprising a His tag (6×His) at the C-terminus) were expressed with Expi293™ expression system (Thermo) and purified with Ni-NTA columns (Thermo). Protein concentration was measured with NanoDrop™ Lite spectrophotometer (Thermo) and protein purity was estimated >95% by SDS-PAGE.

Size exclusion chromatography (SEC) was also performed to characterize VH20-OKT3-Fc BiTE. Briefly, 200 μL (1 μg/mL) filtered samples were used for analysis. Superdex® 200 Increase 10/300 GL columns (GE Healthcare, Cat. No. 28990944) were used. The column was calibrated with protein molecular mass standards of ferritin (440 kDa), aldolase (158 kDa), conalbumin (75 kDa), ovalbumin (44 kDa), carbonic anhydrase (29 kDa), and ribonuclease A (13.7 kDa). Protein was eluted by DPBS buffer at a flow rate of 0.5 mL/minute. As seen in FIG. 5B, VH20-OKT3-Fc is a monomer.

The ability of the VH20-OKT3-Fc BiTE to kill ALK positive cells was also tested. The BiTE mediated cytotoxicity assay was performed by adding a 3-fold serial dilution of the BiTE into culture medium in 96 well cell culture plates. Activated Pan-T cells (Effector cell), and targeted cells (293T, 293T-ALK, SK-N-AS, IMR32, SY5Y) mixtures were added into each well with E:T ratio=4 (10,000 target cells/well). An Iso type BiTE was used as a negative control. After 24 hours of incubation, cytotoxicity was determined by detecting specific lactate dehydrogenase (LDH) released into the medium from the target cells with a cytotoxicity LDH detection kit (Promega). Percent cytotoxicity was calculated with the following formula: Cytotoxicity (%)=(Experimental lysis−Effector spontaneous lysis−Target spontaneous lysis)/(Target maximum lysis−Target spontaneous lysis)×100%. As seen in FIG. 5C, BiTE mediates cytotoxicity to ALK positive tumor cells (293T-ALK, SY5Y, IMR32). The BiTE was not cytotoxic against SK-N-AS cells (negative control). Thus, the VH20-OKT3-Fc BiTE was cytotoxic against different tumor cells (293T-ALK, SY5Y, IMR32 cells, E:T=4:1) with high potency (IC₅₀=0.01 nM for 293T-ALK; 0.15 nM for SY5Y; and 0.1 nM for IMR32).

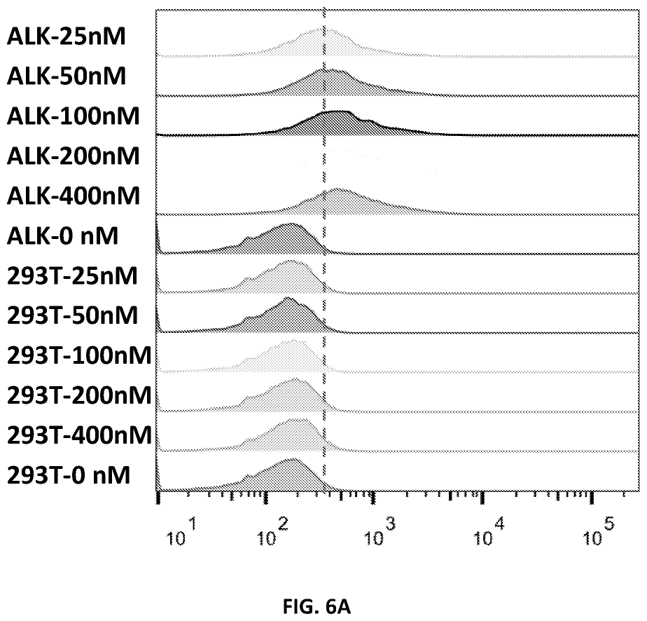

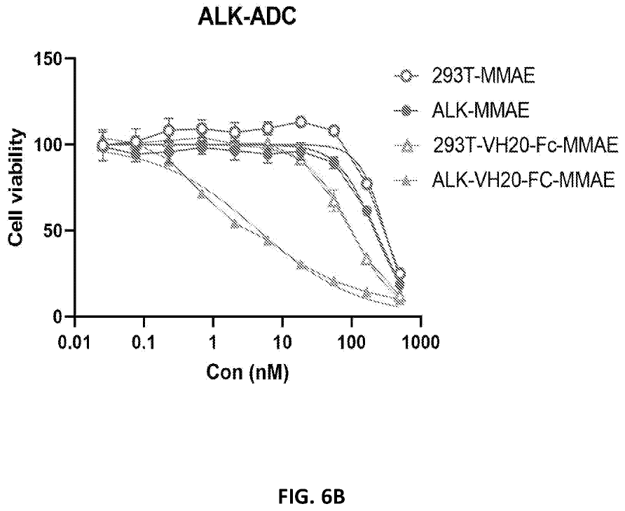

VH20 internalization was also characterized. For the internalization studies, VH20 was conjugated with pHrodo™ Deep Red dye using the pHrodo™ Deep Red Antibody Labeling Kit (Thermo). The conjugated VH20 was incubated with 293T and 293T-ALK cells for 24 hours at different concentrations. VH20 internalization was measured by a flow cytometer. As seen in FIG. 6A, VH20 was effectively internalized into 293T-ALK cells, while no non-specific internalization was detected in 293T cells.

The cytotoxicity of an antibody-drug conjugate (ADC) comprising VH20 was also studied. Specifically, a VH20-Fc-monomethyl auristatin E (MMAE) construct was synthesized. The VH20-ADC was 3-fold serial diluted in cell culture medium in 96 wells. 293T and 293T-ALK cells were added into each well (2000 cells/well), after 4 days of culture. Cell viability was then determined by CellTiter-Glo® Luminescent Cell Viability Assay (Promega). As seen in FIG. 6B, VH20-Fc-MMAE showed potent cytotoxicity (IC₅₀=4.528 nM) against 293T-ALK cells.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. An isolated antibody or antigen binding fragment thereof immunologically specific for anaplastic lymphoma kinase (ALK), wherein said antibody or fragment thereof specifically binds amino acids 733-960 or amino acids 935-1038 of ALK.

2. The antibody or antigen binding fragment thereof of claim 1, wherein said antibody or antigen binding fragment thereof comprises at least one complementarity determining region from VH20 or VH78.

3. The antibody or antigen binding fragment thereof of claim 1, wherein said antibody or antigen binding fragment thereof comprises all three complementarity determining regions from VH20 or VH78.

4. The antibody or antigen binding fragment thereof of claim 1, wherein said antibody or antigen binding fragment thereof comprises a heavy chain comprising GFTFSSYA (SEQ ID NO: 3), IWYDGSNO (SEQ ID NO: 4), and AKSSYYDSSGYYFPYGY (SEQ ID NO: 5).

5. The antibody or antigen binding fragment thereof of claim 1, wherein said antibody or antigen binding fragment thereof comprises a heavy chain comprising GFTFSSYA (SEQ ID NO: 3), IYPGDSDT (SEQ ID NO: 7), and ARERGWQDAFDI (SEQ ID NO: 8).

6. The antibody or antigen binding fragment thereof of claim 1, wherein said antibody or antigen binding fragment thereof comprises a heavy chain comprising
(SEQ ID NO: 2)
EVQLVESGGGVVQPGRSLRLSCAASGFTFSSYAMHWVRQAPGKGLEWVAA
IWYDGSNQYYADSVKGRFTISRDDSTNTLYLQMNSLRAEDTALYYCAKSS
YYDSSGYYFPYGYWGQGTLVTVSS.

7. The antibody or antigen binding fragment thereof of claim 1, wherein said antibody or antigen binding fragment thereof comprises a heavy chain comprising
(SEQ ID NO: 6)
EVQLVESGGGVVQPGRSLRLSCAASGFTFSSYAMHWVRQAPGKGLEW
MGIIYPGDSDTRYSPSFQGHVTISRDDSTNTLYLQMNSLRAEDTATY
YCARERGWQDAFDIWGQGTLVTVSS.

8. An immunoconjugate comprising the antibody or antigen binding fragment thereof of claim 1.

9. An antibody-drug conjugate (ADC) comprising the antibody or antigen binding fragment thereof of claim 1.

10. A chimeric antigen receptor (CAR) comprising the antibody or antigen binding fragment thereof of claim 1.

11. A CAR T-cell comprising the CAR of claim 10.

12. A bispecific T-cell engager comprising the antibody or antigen binding fragment thereof of claim 1.

13. A method for treating, inhibiting, and/or preventing a cancer in a subject comprising administering a composition comprising an anti-ALK antibody or antigen binding fragment thereof comprising compound of claim 1 and at least one pharmaceutically acceptable carrier.

14. The method of claim 13, wherein said cancer expresses ALK on its surface.

15. The method of claim 13, wherein said cancer is neuroblastoma.

16. The method of claim 13, further comprising administering an ALK inhibitor and/or chemotherapeutic agent.

17. The antibody or antigen binding fragment thereof of claim 4, wherein said heavy chain comprises an amino acid sequence having at least 90% identity with SEQ ID NO: 2.

18. The antibody or antigen binding fragment thereof of claim 5, wherein said heavy chain comprises an amino acid sequence having at least 90% identity with SEQ ID NO: 6.
